(19) United States
(12) Patent Application Publication
(10) Pub. No.: US 2005/0149096 A1
(43) Pub. Date: Jul. 7, 2005

(54) Catheter with conduit traversing tip

(76) Inventors: Said S. Hilal, Coto de Caza, CA (US); Matthew N. Petrime, Los Angeles, CA (US); Charles C. Hart, Summerville, SC (US)

Correspondence Address: Richard L. Myers, 22872 Avenida Empresa, Rancho Santa Margarita, CA, 92688, US

(21) Appl. No.: 10/745,262

(22) Filed: Dec. 23, 2003

(51) Int. Cl. A61M 29/00

(52) U.S. Cl. 606/191

(57) ABSTRACT

A catheter facilitating traversal of restrictions in body conduits includes a shaft having a distal tip with a shape that is non-conical, radially twisted, and rectangular in radial cross section. An outer surface of the tip includes at least one side section extending from a blunt point radially outwardly with progressive positions proximally along an axis of the tip. The side section includes a proximal portion in proximity to the shaft, and a distal portion twisted radially with respect to the proximal portion. The catheter can be adapted for placement over a guidewire and can be made transparent thereby facilitating visualization through an endoscope in the catheter.

### BACKGROUND OF THE INVENTION

1. Field of the Invention

This invention relates generally to catheters and other surgical instruments which are required to traverse body conduits.

2. Discussion of Related Art

Catheters are commonly used to traverse body conduits in order to reach distal locations within the conduit. For example, catheters are used to traverse blood vessels and ureteral conduits, and endoscopes are used to traverse intestinal conduits.

Traversing a particular conduit can often be difficult, particularly where there are restrictions within the conduit. These restrictions can be caused by blockages in the form of plaque in the case of blood vessels and strictures in the case of ureteral passages.

In a more specific example, the use of catheters for ureteral access typically encounters a significant obstruction or restriction in perhaps 15% of the cases. In the past, these restrictions have been traversed using dilators to enlarge the ureter passage before the catheter is even inserted. Repeated dilation with dilators of increasing size is often required.

In the past, catheters have typically been provided with conical tips which taper proximally from a point. This shape has been found to be less than optimal in traversing restrictions within a body conduit. In fact, the conical shape appears to be one of the least favorable shapes for this application.

### SUMMARY OF THE INVENTION

In accordance with the present invention, a catheter such as an access sheath, can be inserted into a body conduit using an obturator with a specially formed tip. Rather than attempting to dilate a sphincter or stricture using a conical tip, the present invention contemplates a non-conical tip configuration.

Using a non-conical tip configuration, the obturator can be guided around this stricture and then used to dilate the conduit for the following catheter. An axial force can be applied to the non-conical tip with perhaps the addition of a radial twisting force. With a non-conical tip, this force is directed against a smaller area of the stricture or other restriction. In this manner, the same force applied to a smaller area result in a greater pressure and therefore facilitates dilation of the body conduit.

These and other features and advantages of the invention will become more apparent with a discussion of preferred embodiments and reference to the associated drawings.

### DESCRIPTION OF THE DRAWINGS

FIG. 1 is a side elevation view of a patient having a blood vessel operatively accessed with a catheter system of the present invention;

FIG. 2 is an enlarged side view of the catheter system including an access sheath and an obturator with a blunt tip;

FIG. 3 is a radial cross section view taken along lines 3-3 of FIG. 2;

FIG. 4 is a radial cross section view taken along lines 4-4 of FIG. 2;

FIG. 5 is a perspective view of a preferred embodiment of the obturator tip illustrated in FIG. 2;

FIG. 6 is a side elevation view of the obturator tip taken along lines 6-6 of FIG. 5;

FIG. 7 is a side elevation view taken along lines 7-7 of FIG. 6;

FIG. 8 is an end view taken along lines 8-8 of FIG. 6;

FIG. 9 is a radial cross-section view taken along line 9-9 of FIG. 6;

FIG. 10 is a radial cross-section view taken along line 10-10 of FIG. 6;

FIG. 11 is a radial cross-section view taken along lines 11-11 or FIG. 6;

FIG. 12 is a radial cross-section view taken along lines 12-12;

FIG. 13 is a radial cross-section view taken along lines 13-13 of FIG. 6;

FIG. 14 is a schematic view illustrating each of the Figures of 5-10 super-imposed to facilitate an understanding of the twisted configuration of the blunt tip; and

FIG. 15-40 show perspective views of other embodiments of the blunt tip of the present invention.

### DESCRIPTION OF PREFERRED EMBODIMENT AND BEST MODE OF THE INVENTION

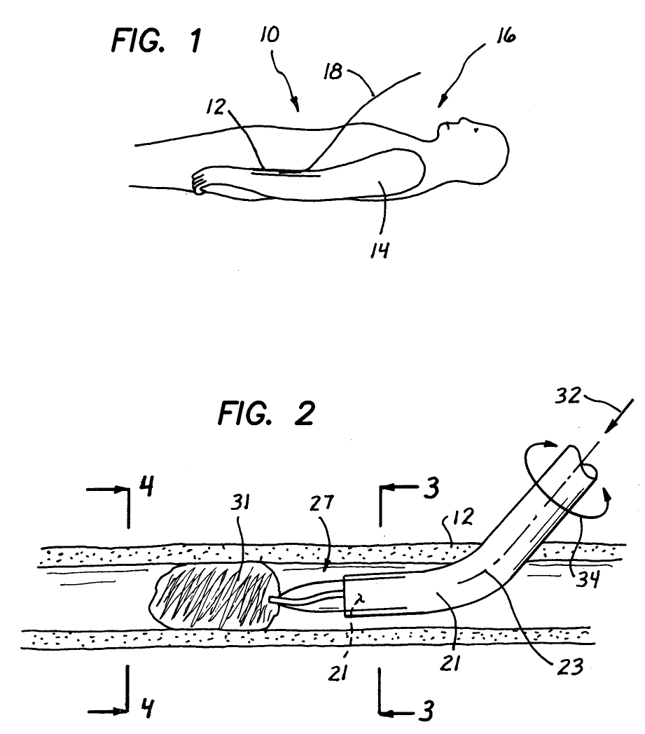

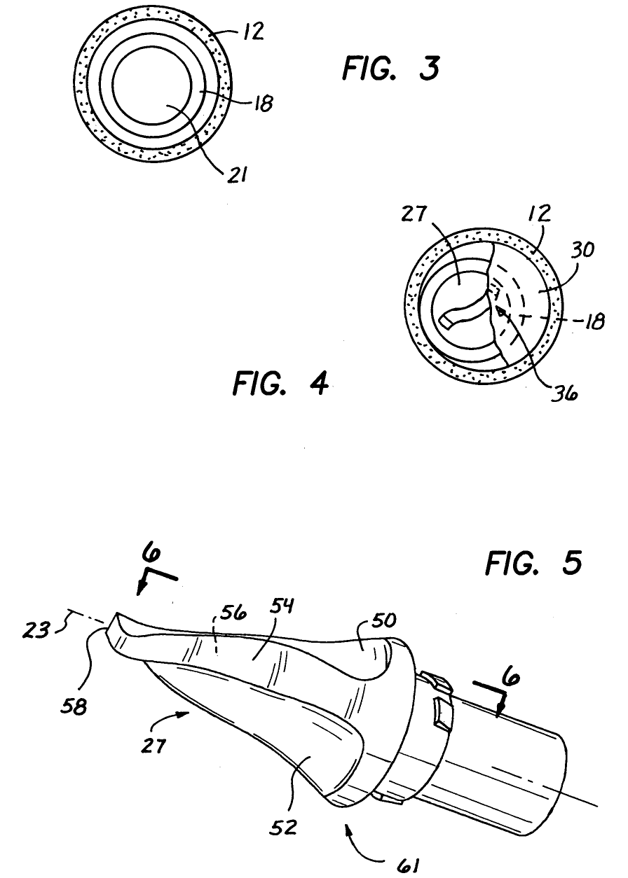

A catheter system is illustrated in FIG. 1 and designated by the reference numeral 10. In this case, the catheter system is illustrated to be operatively disposed to provide access to a blood vessel 12 in the arm of a patient 14. In this case, the catheter system 10 includes an access catheter or sheath 18 and associated obturator 20.

The obturator 20 includes a shaft 21 having a diameter slightly smaller than the inside diameter of the access sheath 18. This shaft 21 has an axis 23 which extends between a proximal handle 25 and a distal tip 27.

It is the distal tip 27 that is of particular interest to the present invention. In comparison to the conical tip configurations of the past, it will initially be noted that the distal tip 27 in this embodiment has a generally blunt configuration and is twisted about the axis 23.

In order to fully appreciate the various aspects of this construction, it is helpful to initially discuss the anatomy associated with typical body conduits such as blood vessels and the urinary tract. It is not uncommon in these body passages for restrictions to develop along the inner wall of the conduit. These restrictions may be natural in the case of a sphincter in the urinary tract, or may develop from various and random causes in the case of strictures in the urinary tract, and blood cots and plaque in the case of blood vessels. In all cases, the restrictions reduce the interior diameter of the conduit making it difficult to traverse through the conduit, for example, with the access sheath 18.

In the past, in order to facilitate traversal of a restriction, a guidewire initially has been passed through the conduit. Then, an obturator has been disposed within the access sheath and directed along the guidewires with the conical obturator tip extending beyond the access sheath 18. An axial force has then been applied in an effort to traverse the restriction.

Since the conical configuration of the distal tip encounters resistance around its entire radial circumference, it is now apparent that this conical structure of the past is one of the least advantageous designs for traversing a restriction.

In FIG. 2, the catheter system 10 of the present invention is illustrated to be placed within the vessel 12 with the distal tip 27 encountering a restriction 30. The catheter system in this embodiment is provided with a guidewire lumen 11 and otherwise adapted for placement over a guidewire 13. At this point, an axial force, represented by an arrow 32, as well as a twisting force, represented by an arrow 34, can be applied to the shaft 21 of the obturator 20. With the blunt and twisted configuration of the distal tip 27, contact is made with the restriction 30 at a very small area shown generally by the reference numeral 36 in FIG. 4. With this small area of contact 36, the axial force 32 and twisting force 34 can exert a high pressure against the restriction 30 in order to facilitate dilation of the vessel 12 and passage of the restriction 30.

The twisted configuration of the tip 27 also causes the tip 27 to function with the mechanical advantage of a screw thread. With this configuration, a preferred method of placement requires that the user grip the sheath 18, and twist it about the axis 23. This twisting motion in combination with the screw configuration of the tip 27 converts radial movement into forward movement along the axis 23. Thus, the user can apply both a forwardly directed force as well as a radially directed force to move the catheter system 10 in a forward direction.

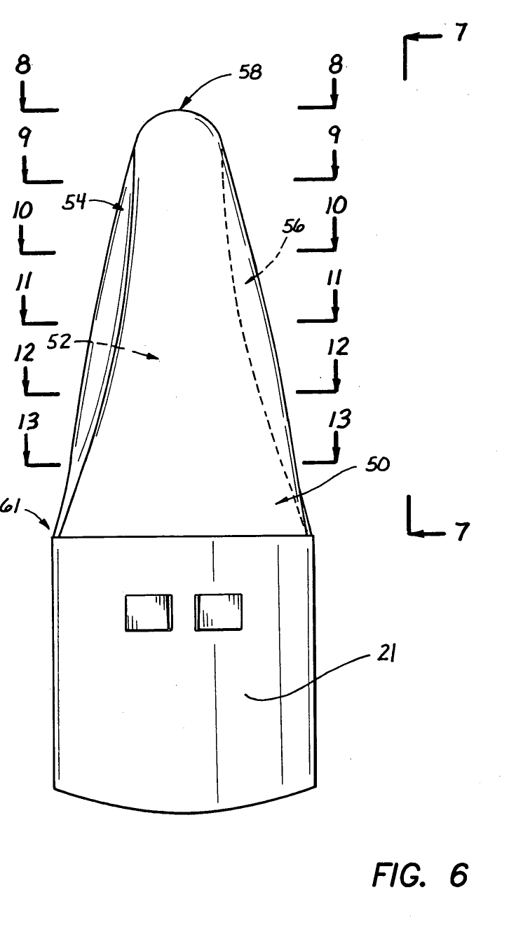

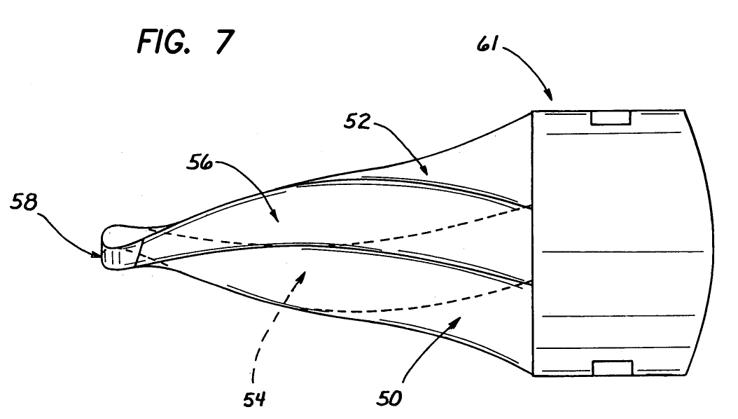

The twisted and rectangular configuration of the tip 27 is most apparent in the schematic view of FIG. 5 and the side views of FIGS. 6 and 7. In this embodiment, the tip 27 is composed generally of four surfaces: two opposing major surfaces 50 and 52, separated by two side surfaces 54 and 56 which extend between an end surface 58 and a proximal base 61. A plane drawn through the axis 23 would show the tip 27 in this case, to be composed of two symmetrical halves.

The major surfaces 50 and 52 and the side surfaces 54 and 56 generally define the cross section of the tip 27 to be rectangular from the end surface 58 to the proximal base 61. This configuration can best be appreciated with reference to the cross section views of FIGS. 8-13. In FIG. 8, the distal end of the tip 27 is shown as a rectangle having its greatest length-to-width ratio. This rectangle, designated by the reference numeral 63, also has a twisted S-shaped configuration at the distal-most end of the tip 27.

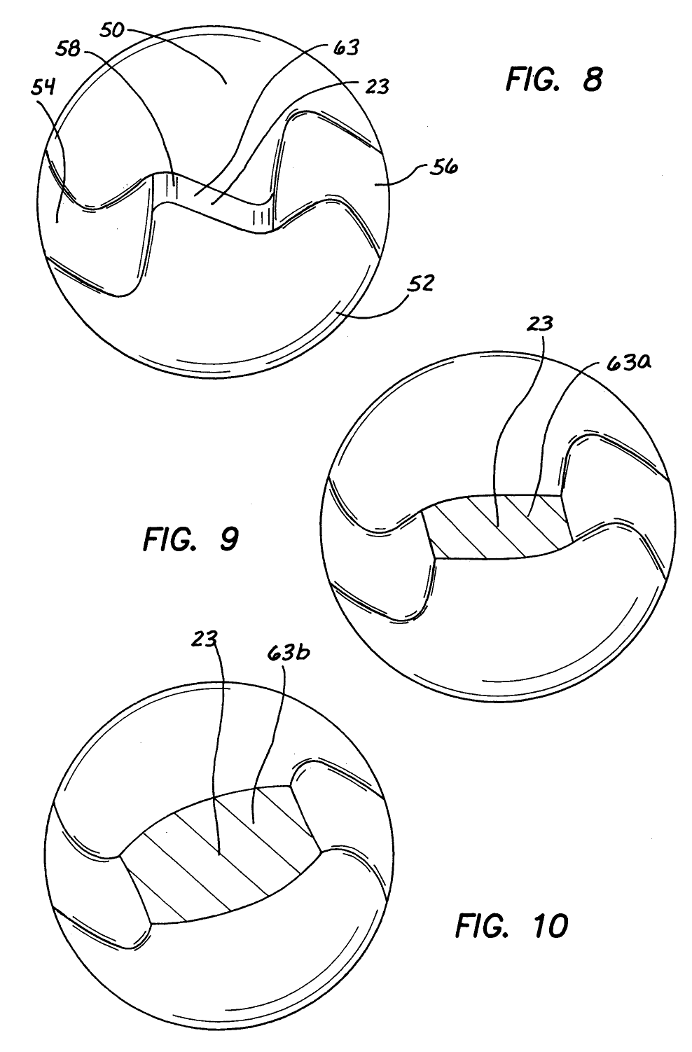

As views are taken along progressive proximal cross sections, it can be seen that the rectangle 63 becomes less twisted, and the width increases relative to the length of the rectangle 63. The spiral nature of the tip 27 is also apparent as the rectangle moves counterclockwise around the axis 23 in the embodiment of FIG. 5. This is perhaps best appreciated in a comparison of the rectangle 63 in FIG. 10 relative to that in FIG. 9. With progressive proximal positions, the rectangle 63 begins to fatten with a reduction in the ratio of length to width. The long sides of the rectangle 63 also tend to become more arcuate as they approach a circular configuration most apparent in FIGS. 12 and 13. In these figures, it will also be apparent that the rotation of the rectangle 63 reaches a most counterclockwise position and then begins to move clockwise. This is best illustrated in FIGS. 11, 12 and 13. This rotation back and forth results from the configuration of the side surfaces 54 and 56, which in general, have a U-shape best illustrated in FIGS. 5 and 6.

The ratio of the length-to-width of the rectangle 63 is dependent on the configuration of the side surfaces 54 and 56, which defined the short sides of the rectangle 63, as well as the configuration of the major surfaces 50 and 52 which define the long sides of the rectangle 63. Again with reference to FIG. 8, it can be seen that the side surfaces 50 and 52 are most narrow at the distal end of the tip 27. As these surfaces extend proximally, they reach a maximum width near the point of the most counterclockwise rotation, shown generally in FIG. 11, and then reduce in width as they approach the proximal base 61. Along this same distal to proximal path, the major surfaces 50 and 52 transition from a generally flat configuration at the distal end to a generally conical configuration at the proximal end 61.

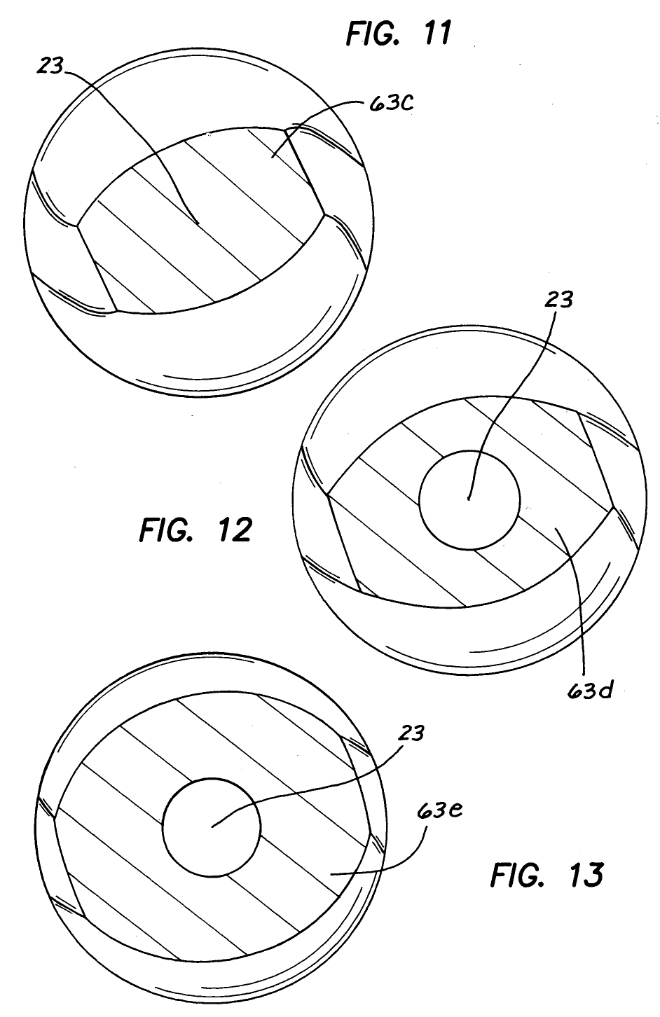

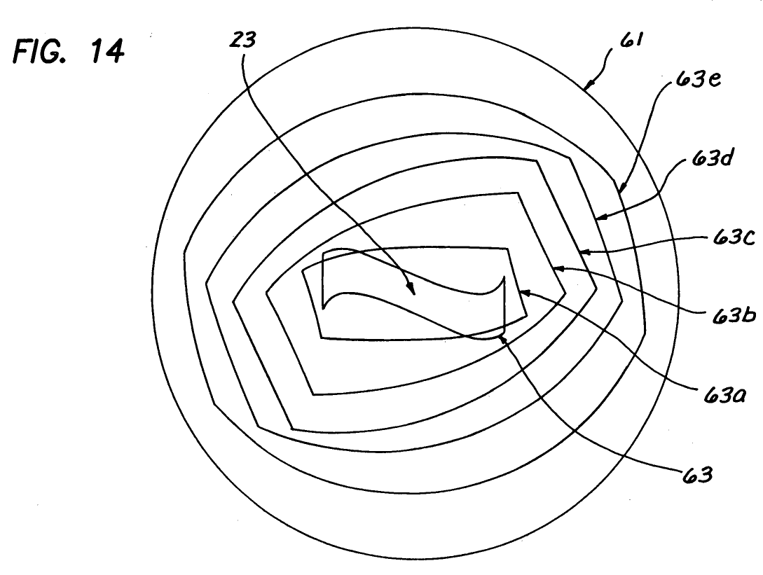

In the progressive views of FIGS. 9-13, the rectangle 63 is further designated with a lower case letter a, b, c, d, or e, respectively. In FIG. 14, the rectangles 63 and 63a-63c are superimposed on the axis 23 to show their relative sizes, shapes, and angular orientations.

A preferred method of operating the catheter system 10 benefits significantly from this preferred shape of the blunt tip 27. With a rectangular configuration at the distal surface 58, the end of the tip 27 appears much like a flathead screwdriver. With this shape, the simple back and forth twisting motion tends to open the vessel 12 to accept the larger diameter of the sheath 18. Again, a twisting or dithering motion facilitates transversal of the restriction 30, thereby requiring a significantly reduced penetration force along the arrow 34. This process continues with safety and ease until the device passes the restriction 30 and moves on through the conduit or vessel 12.

The obturator 20 can be constructed as a single component or divided into two components such as the shaft 21 and the tip 27. If the obturator 20 is constructed as a single component, it may be formed of either disposable or reusable materials. If the obturator 18 is constructed as two or more components, each component can be made either disposable or useable as desired for a particular configuration. In certain preferred embodiments, the obturator shaft 21 and handle are made of a reusable material, such as a metal or an autoclavable polymer in order to facilitate re-sterilization and reuse of these components. In this embodiment, the tip 27 is made of a material that is not autoclavable and therefore is adapted to be disposable.

The blunt tip 27 can be coated or otherwise constructed from a soft elastomeric material. In such a case, the material could be a solid elastomer or composite elastomer/polymer.

The shaft 21 of the obturator 20 can be partially or fully flexible. With this configuration, the obturator 20 could be inserted through a conduit containing one or more curves of virtually any shape. A partially or fully flexed obturator 18 could be used with a flexible sheath 18 allowing greater conformity to the shape of the conduit.

The obturator 18 could also be used as an insufflation needle and provided with a passageway and valve to administer carbon dioxide or other insufflation gas to the peritoneal cavity 32. The obturator 18 could also be used with an insufflation needle cannula, in which cases removal of the obturator 18 upon entry would allow for rapid insufflation of the peritoneal cavity 32.

The obturator 18 could also be constructed to permit free spinning of the tip about the axis 23. This would allow the tip 27 to find its own way around the restriction 30 rather than relying on the user for clockwise and counterclockwise rotation.

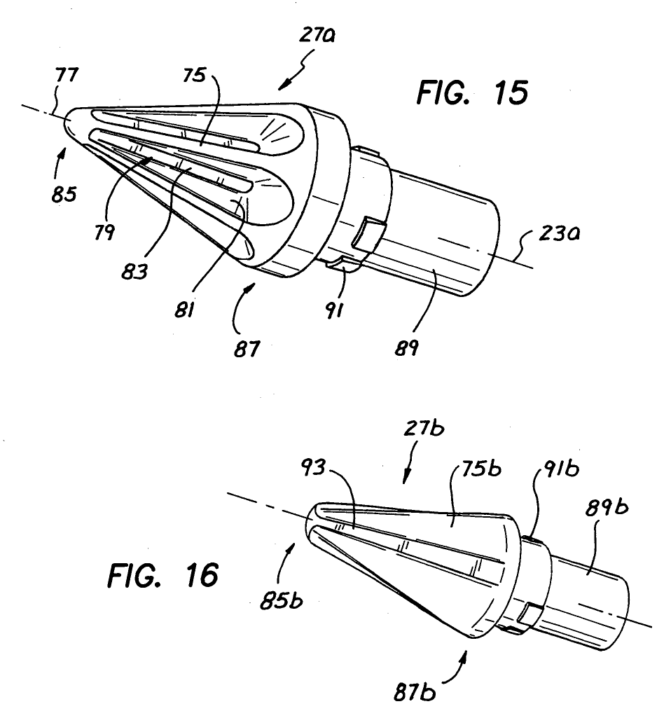

Other embodiments of the invention are illustrated in FIG. 12-37 where elements of structure similar to those previously disclosed are designated with the same reference numeral followed by the lower case letters “a” to “z”, respectively. Thus, in FIG. 15, the tip 27 is referred to with the reference numeral 27a while in FIG. 38, the tip is referred to with a reference numeral 27z.

In FIG. 15, the obturator tip 27a is formed with a conical surface 75 having an axis 77. In this embodiment, the axis 77 of the surface 75 is colinear with the axis 23a of the tip 27a. A plurality of recesses 79 are formed in the conical surface 75 around the axis 77. These recesses are formed with side walls 81 which extend radially inwardly to a valley 83. In this embodiment, the conical surface 75 has an angle with respect to the axis 77 which is greater than an angle between the valley 83 and the axis 77. As a result, the recesses 79 appear to deepen relative to the surface 75 from a distal end 85 to a proximal end 87 of the tip 27a. The sidewalls 81 have a generally constant angle with respect to the conical surface 75 and consequently have an increased area toward the proximal end 87. The valley 83 has a generally constant width as it extends towards the proximal end 87.

In this embodiment, the tip 27a also has a cylindrical mounting shaft 89 with mounting lugs 91. This mounting shaft 89 is adapted to closely fit within the obturator shaft 21 (FIG. 1). The mounting lugs 91 can engage holes or shoulders within the shaft 21 to facilitate a fixed but removable relationship between the shaft 21 and tip 27a.

In FIG. 16, the tip 27b is also characterized by the conical surface 75b, the cylindrical mounting shaft 89 and the lugs 91b. In this case, the tip 27b is provided with ridges 93 which extend radially outwardly from the conical surface 75b. The ridges 93 can have a constant width or a width which increases proximally as in the illustrated embodiment. The height of the ridges above the conical surface 75b can be either constant or variable between the distal end 85band the proximal end 87b.

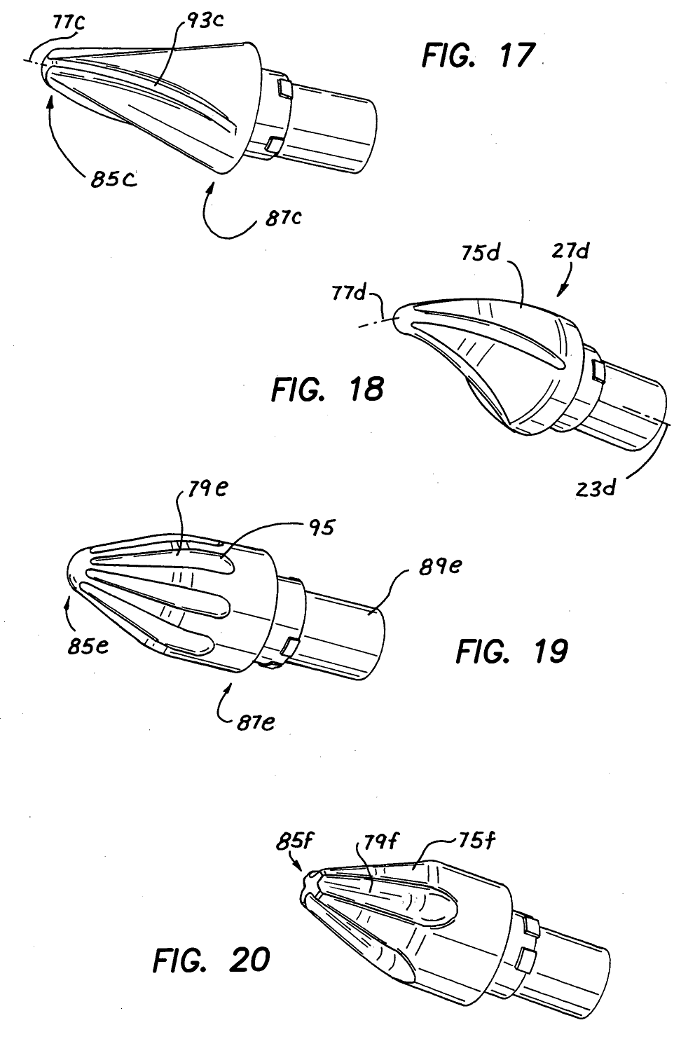

The obturator tip 27c in FIG. 17 is similar to that of FIG. 13 except that the ridges 93c are not straight but rather curved as they extend between the distal end 85c and the proximal end 87c. In this case, the ridges have an angle with respect to the axis 77c which increases proximally both radially and axially.

The obturator tip 27d in FIG. 18 is similar to that of FIG. 15 except that the axis 77d of the conical surface 75d is curved rather than straight. Accordingly, the axis 77d of the conical surface 75d is curved relative to the axis 23d of the obturator shaft 21d.

The obturator tip 27e in FIG. 19 is similar to that of FIG. 15 in that it includes the recesses 79e which extend from the distal end 85e to the proximal end 87e. In this case however, the tip 27e has a cylindrical surface 95 which extends proximally of the conical surface 75e between the distal tip 85e and the mounting shaft 89e. The recesses 79e in this embodiment extend along both the conical surface 75e and the cylindrical surface 95.

The obturator tip 27f of FIG. 20 is similar to that of FIG. 19 except that the recesses 79f extend through the distal end 85f. In the illustrated embodiment, four of the recesses 79f provide the distal end 85f with the shape of the letter “X.”

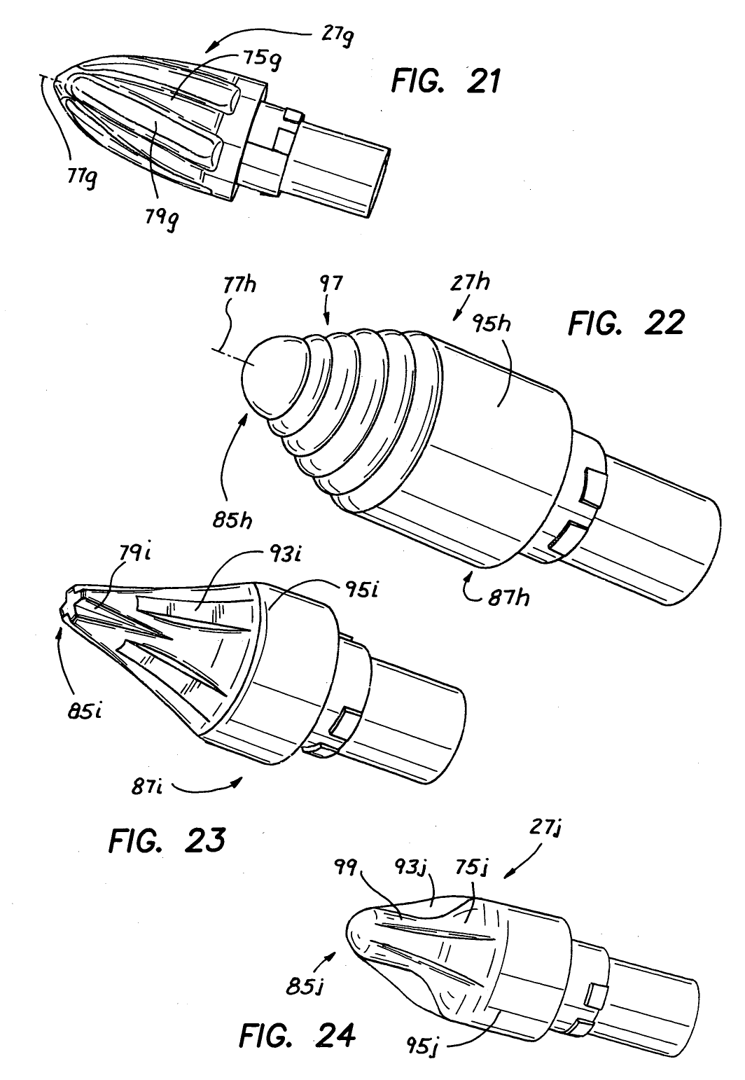

The obturator tip 27g in FIG. 21 is similar to that of FIG. 15 except that the surface 75g is more rounded thereby providing the tip 27g with a parabolic or bullet shape. Also, the recesses 79g are disposed at an angle with respect to any plane passing through the axis 77g.

The obturator tip 27h in FIG. 22 has the cylindrical surface 95h at its proximal end 87h and a series of grooves 97 which extend circumferentially of the axis 77h with diameters which increase from the distal end 85h to the cylindrical surface 95h. Each of the recesses or ridges in the series 97h is disposed in an associated plane that is perpendicular to the axis 77h.

In the embodiment of FIG. 23, the tip 27i includes recesses 79i which are similar to those illustrated in FIG. 20 in that they extend through the distal end 85i. This embodiment also includes the ridges 93i which are disposed between the recesses 79i and extend toward the cylindrical surface 95i at the proximal end 87i. The recesses 79i in FIG. 23 have individual widths which decrease proximally.

In the embodiment of FIG. 24, the tip 27j includes the conical surface 75j which transitions proximally into the cylindrical surface 95j. Distally of the conical surface 75j a second cylindrical surface 99j is provided which extends to the distal end 85j. Ridges 93j extend radially outwardly from the second surface 99 and the conical surface 75j.

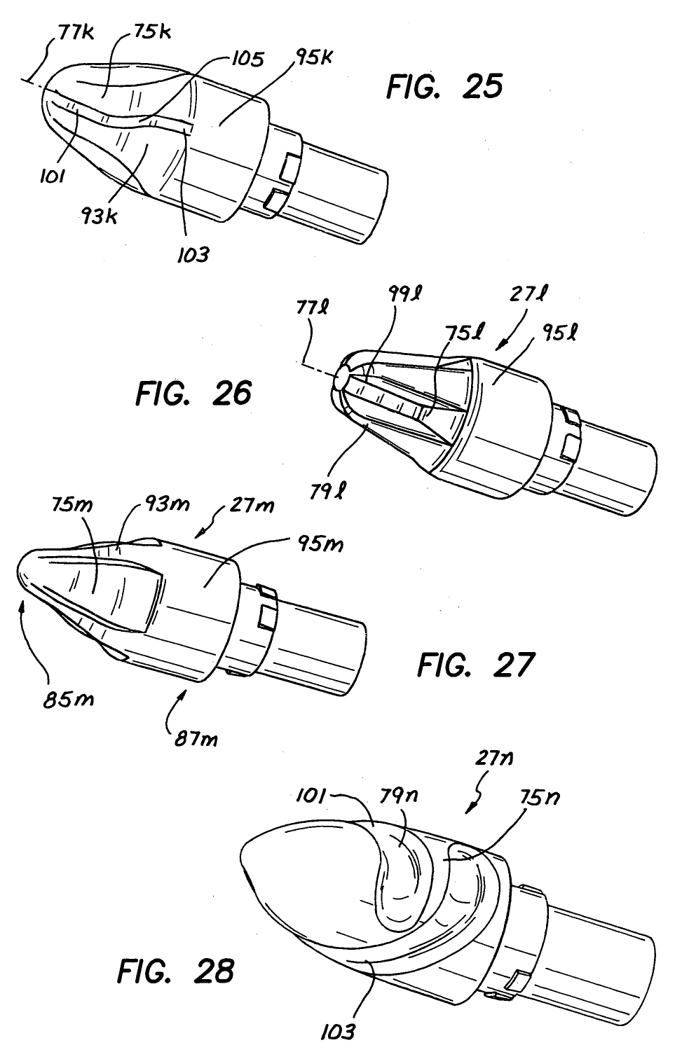

The obturator tip 27k in FIG. 25 is similar to previous embodiments having the conical surface 75k and the cylindrical surface 95k. In this embodiment, the ridges 93k include distally portions 101 and proximal portions 103 which extend in planes passing through the axis 77k. Between the proximal portions 103 and distal portions 101, the ridges 93k include intermediate portions 105 which extend in planes that do not include the axis 77k.

In FIG. 26, the tip 27L is similar to that of FIG. 20 except that the second cylindrical surface 99L is provided in this embodiment. The recesses 79L have a generally constant width along the second cylindrical surface 99L and the conical surface 75L. These recesses 79L do not extend into the cylindrical surface 95L.

The obturator tip 27m in FIG. 27 is similar to that of FIG. 24 except that it does not include the second cylindrical surface 99m. In this case, the conical surface 75m extends to the distal end 85m with a slightly concave shape. The ridges 93m transition into the surface 75m at the distal end 85m and transition into the cylindrical surface 95m at the proximal end 87m. Between these two ends, the ridges 93m have a height which is increased by the concave configuration of the surface 75m.

The tip 27n in FIG. 28 is similar to the tip 27g in FIG. 21 in that the outer surface 75n has a generally bullet-shaped configuration. The recesses 79n include a recess 101 which curves proximally in a counterclockwise direction, and a recess 103 which curves proximally in a clockwise direction.

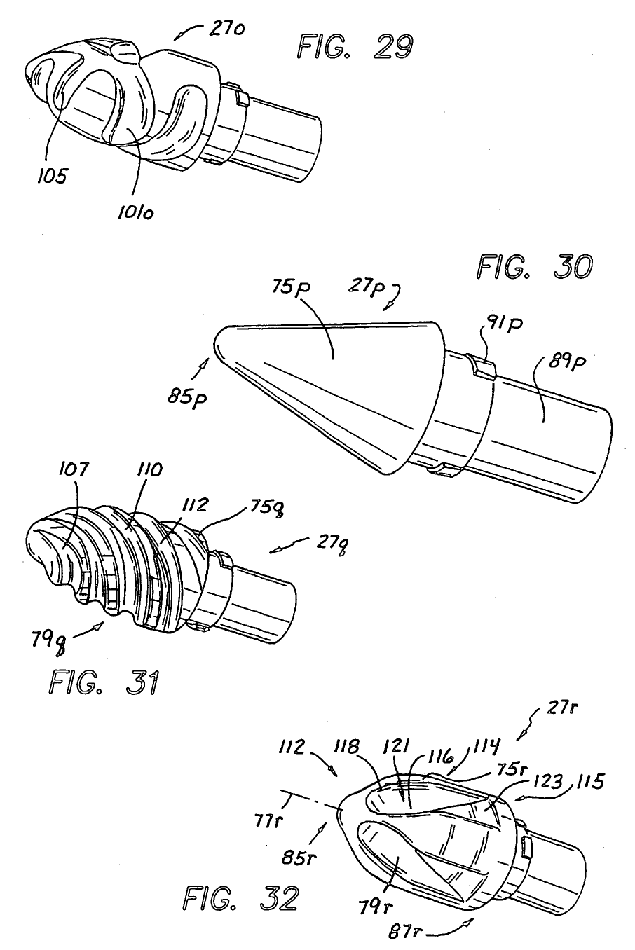

The tip 27o in FIG. 29 is similar to that of FIG. 28 but includes a further recess 106 which spirals toward the distal end 85o in a clockwise direction. This spiral recess 106 crosses the recess 101o in this embodiment.

In FIG. 30, the tip 27p includes the conical surface 75p which extends toward the distal end 85p at its apex. The apex of the outer conical surface 75p is blunted at the distal end 85p. This embodiment also includes the mounting shaft 89p and associated lugs 91p.

The tip 27q in FIG. 31 has the outer surface 75q with a bullet-shaped configuration. The recesses 79q in this embodiment include three recesses, 107, 110, and 112 which spiral in a generally parallel relationship proximally in a counterclockwise direction.

The tip 27r in FIG. 32 has an outer surface 75r with a bullet-shaped configuration, and a plurality of recesses 79r which extend generally axially from the distal end 85r to the proximal end 87r. The recesses 79r are generally axially symmetrical and include a proximal portion 113, and a distal portion 114 with sidewalls 116 and 118 which define a deep valley 121 that extends generally parallel to the axis 27r. The proximal portion 113 of the recess 79r comprises a plane 123 which extends between the sidewalls 118 and 121 from the valley 121 radially outwardly with progressive positions toward the proximal end 87r.

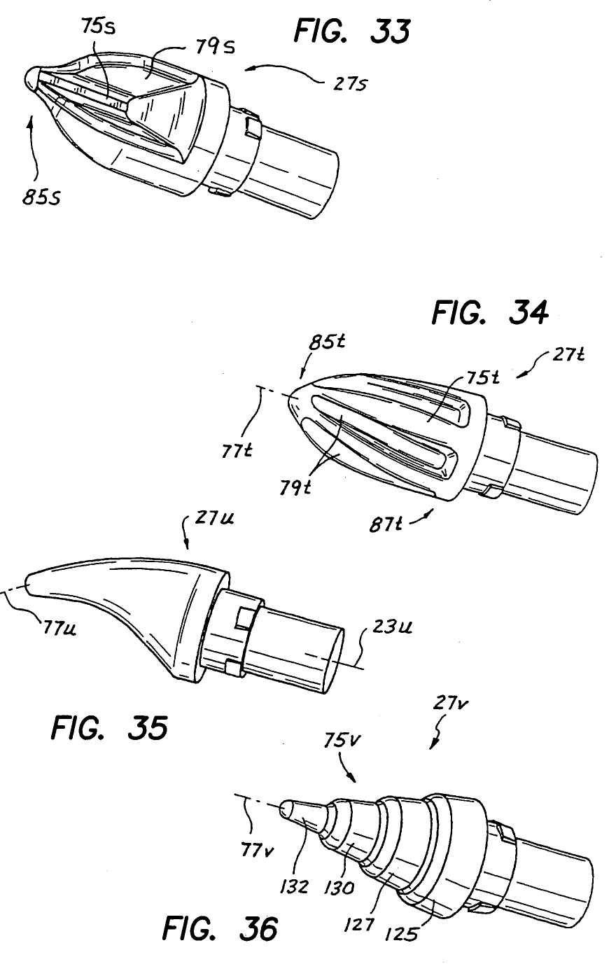

The tip 27s in FIG. 33 is similar to that of FIG. 32, but includes fewer recesses 79s. Also, the tip 27s has a nose that is more pointed thereby providing the outer surface 75s with a concave configuration near the distal end 85s.

FIG. 34 shows a perspective view of the tip 27t with a bullet-shaped outer surface 75t and a plurality of the recesses 79t. In this case the recesses are straight but nevertheless have an angular relationship with the axis 77t. These recesses 79t extend through the distal end 85t but stop short of the proximal end 87t.

The tip 27u in FIG. 35 is similar to that of FIG. 18 in that the axis 77u is curved relative to the axis 23u which is straight. Also, in this embodiment, there are no ridges or recesses.

In FIG. 36, the tip 27v has an outer surface 75v which is formed by individual frustoconical portions 125, 127, 130, and 132, which have progressively smaller average diameters. These conical portions 125-132 appear to be stacked with their individual axes disposed along the common axis 77v.

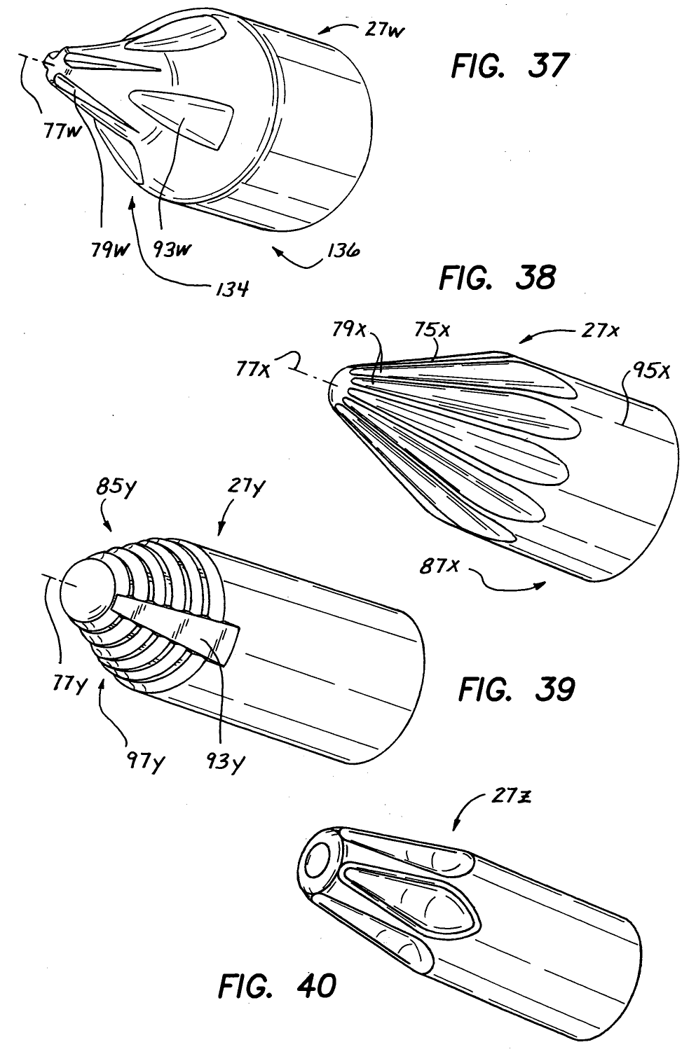

The tip 27w in FIG. 37 is similar to that of FIG. 23 in that it includes both the recesses 79w, as well as the ridges 93w. In this embodiment, which includes both a distal portion 134, as well as a proximal portion 136. These portions 124 and 136 have a generally common dimension along the axis 77w.

The tip 27x in FIG. 38 includes the conical surface 75x as well as the cylindrical surface 95x. The recesses 79x are oriented generally in respective radial planes. These recesses 79x are similar in shape and have a width which increases toward the distal end 87x.

The tip 27y in FIG. 39 is similar to that of FIG. 22. It includes concentric circular structures at the distal end 85y. In this case however, the structures are a series of recesses 97y rather than ridges. This embodiment includes at least one ridge 93y, however, which extends radially outwardly with progressive proximal positions along the axis 77y.

The tip 27z in FIG. 40 is similar to that of FIG. 38 except that it includes recesses 79z which are fewer in number but wider in size. Also, the nose of the tip 27 and at the distal end 85z is accentuated in the embodiment of FIG. 40′.

A feature which may be of particular interest to any of these embodiments, relates to illumination and visualization properties of the tip 27. In a preferred embodiment, such as that illustrated in FIG. 2, a source of illumination and/or a scope can be inserted into a lumen, similar to the guidewire lumen 11, to facilitate visualization of the operative site. In such an embodiment, the tip 27 is preferably made of a transparent plastic material.

It will be understood that many modifications can be made to the various disclosed embodiments without departing from the spirit and scope of the concept. For example, various sizes of the surgical device are contemplated as well as various types of constructions and materials. It will also be apparent that many modifications can be made to the configuration of parts as well as their interaction. For these reasons, the above description should not be construed as limiting the invention, but should be interpreted as merely exemplary of preferred embodiments. Those skilled in the art will envision other modifications within the scope and spirit of the present invention as defined by the following claims.

1. A surgical catheter adapted to traverse a restriction within a body conduit, comprising:
an elongate flexible shaft extending along an axis between a proximal end and a distal end;
a tip disposed at the distal end of the shaft the tip having an outer surface extending distally to a blunt point;
the outer surface having at least one section extending from the blunt point radially outwardly with progressive positions proximally along the axis;
the side section including a distal portion in proximity to the blunt point, and a proximal portion in proximity to the shaft; and
the distal portion of the side section being twisted radially with respect to the proximal portion of the side section.

2. The surgical catheter recited in claim 1, wherein the side section is a first side section and the catheter further comprises:
a second side section of the outer surface; and
the second side section being separated from the first side section by at least one intermediate section of the outer surface.

3. The surgical catheter recited in claim 2, wherein the intermediate section extends across the blunt point of the tip.

4. The surgical obturator recited in claim 3, wherein:
the intermediate section includes a distal portion in proximity to the blunt point and a proximal portion in proximity to the shaft;
the distal portion of the intermediate surface being twisted in a first radial direction; and
the proximal portion of the intermediate surface being twisted in a second radial direction opposite the first radial direction.

5. The surgical obturator recited in claim 4, wherein the distal portion of the intermediate section has a width which increases proximally.

6. The surgical obturator recited in claim 5, wherein the proximal portion of the intermediate section has a width which decreases proximally.

7. The surgical obturator recited in claim 1, wherein the outer surface in radial cross section has the general configuration of a geometric shape.

8. The surgical obturator recited in claim 1, wherein the intermediate section of the outer surface separates first and second side sections of the outer surface and extends distally to the blunt point, across the blunt point, and proximally from the blunt point.

9. A surgical catheter adapted to traverse a restriction in a body conduit comprising;
an elongate shaft extending along an axis between a proximal end and a distal end;
a tip disposed at the distal end of the shaft, the tip having an outer surface with a distal portion and a proximal portion;
the outer surface of the tip in radial cross section having the general configuration of a geometric shape with a side;
the side of the geometric shape in the distal portion of the tip rotating in a first direction about the axis with progressive radial cross sections proximally along the axis.

10. The surgical obturator recited in claim 9, further comprising:
the side of the geometric shape in the proximal portion of the tip rotating in a second direction opposite to the first direction with progressive radial cross sections proximally along the axis.

11. The surgical obturator recited in claim 9, wherein the geometric shape is a rectangle.

12. The surgical obturator recited in claim 11, wherein the side is a long side of the rectangle and the rectangle further comprises:
a short side having a length less than that of the long side; and
the ratio of the length of the long side to the length of the short side decreases with progressive radial cross sections proximally along the axis.

13. The surgical obturator recited in claim 11, wherein the rectangle at the point of the tip has the general shape of the letter “S.”

14. A surgical catheter adapted to traverse a body conduit, comprising:
an elongate shaft extending along an axis between a proximal end and a distal end;
a tip having an outer surface including a pair of generally opposed sections;
the outer surface having generally a geometric shape in progressive radial cross sections from a distal cross section to a proximal cross section;
the pair of generally opposed sections of the outer surface appearing as a pair of lines in each of the progressive radial cross sections; and
at least one of the pair of lines becoming increasing arcuate in the progressive radial cross sections.

15. The surgical catheter recited in claim 14, wherein the area of the geometric shape increases along the progressive radial cross sections.

16. The surgical catheter recited in claim 15, wherein:
the geometric shape is a rectangle having a first side with a first length, and a second side with a second length shorter than the first length;
the rectangle having a particular ratio characterized by the first length divided by the second length; and
the particular ratio decreasing along the progressive radial cross sections.

17. The surgical catheter recited in claim 15, wherein the at least one of the pair of lines rotates in a first direction around the axis in the progressive radial cross sections.

18. The surgical catheter recited in claim 17, wherein the at least one line rotates around the axis in a second direction opposite to the first direction in the progressive radial cross sections.

19. A surgical catheter adapted to traverse a body conduit, comprising:
an elongate shaft extending along an axis;
a tip coupled to the shaft and having an axis extending between a proximal end and a distal end, the tip having an outer surface with a generally conical configuration and a blunt tip;
portions of the outer surface of the tip defining at least one recess extending relative to the axis generally between the proximal end and the distal end of the tip.

20. The surgical catheter recited in claim 19, wherein the recess extends generally in a plane common to the axis of the tip.

21. The surgical catheter recited in claim 19, wherein the recess extends generally spirally of the axis of the tip

22. The surgical catheter recited in claim 19, wherein the recess extends to the blunt point of the tip.

23 The surgical catheter recited in claim 22, wherein the blunt point of the bladeless tip has the shape of a cross.

24. The surgical catheter recited in claim 21, wherein:
the recess is a fist recess spiraling relative to the axis in a first direction to the access; and
the portions define a second recess spiraling relative to the axis in a second direction opposite of the first direction.

25. The surgical catheter recited in claim 19, wherein the axis is curved.

26. The surgical catheter recited in claim 1, wherein the tip is formed of a transparent material to facilitate illumination and visualization through the tip of the catheter.

27. The surgical catheter recited in claim 9, wherein the tip is formed of a transparent material to facilitate illumination and visualization through the tip of the catheter.

28. The surgical catheter recited in claim 14, wherein the tip is formed of a transparent material to facilitate illumination and visualization through the tip of the catheter.

29. The surgical catheter recited in claim 19, wherein the tip is formed of a transparent material to facilitate illumination and visualization through the tip of the catheter.
